(19) United States
(12) Patent Application Publication
(10) Pub. No.: US 2021/0174939 A1
(43) Pub. Date: Jun. 10, 2021

(54) DEEP LEARNING SYSTEM FOR DETECTING ACUTE INTRACRANIAL HEMORRHAGE IN NON-CONTRAST HEAD CT IMAGES

(71) Applicant: TENCENT AMERICA LLC, Palo Alto, CA (US)

(72) Inventors: Chao HUANG, Palo Alto, CA (US); Zhen Qian, Santa Clara, CA (US); Hui Tang, Mountain View, CA (US); Yusheng Xie, Mountain View, CA (US); Shih-Yao Lin, Palo Alto, CA (US); Kun Wang, San Jose, CA (US); Xiaozhong Chen, Cedarburg, WI (US); Zhimin Huo, Palo Alto, CA (US); Wei Fan, New York, NY (US)

(73) Assignee: TENCENT AMERICA LLC, Palo Alto, CA (US)

(21) Appl. No.: 16/706,998

(22) Filed: Dec. 9, 2019

(51) Int. Cl. G16H 30/40; G06N 3/04; G16H 50/20; G01R 33/56; A61B 6/00; A61B 6/03; A61B 5/055; A61B 5/00

(52) U.S. Cl. CPC G16H 30/40; G06N 3/04; G16H 50/20; G01R 33/5608; A61B 6/507; A61B 6/501; A61B 6/5223; A61B 5/055; A61B 6/037; A61B 5/0042; A61B 6/032

(57) ABSTRACT

A method, computer program, and computer system is provided for receiving data corresponding to a tomograph scan associated with a patient, extracting slices from the received tomograph scan data, and determining adjacent slices for each of the extracted slices. The extracted slices and the adjacent slices may be grouped into slabs, and features associated with the slabs may be identified. It may be determined that a slab corresponding to the identified features contains a feature associated with ICH.

### BACKGROUND

This disclosure relates generally to field of medicine, and more particularly to detection of intracranial hemorrhage (ICH).

An intracranial hemorrhage (ICH) is a critical condition resulting from bleeding within the skull. ICH accounts for about two million strokes worldwide, and prompt diagnosis is required in order to optimize patient outcomes. Non-contrast computed tomography (CT) scans of a patient's head are used for initial imaging in cases of head trauma or stroke-like symptoms.

### SUMMARY

Embodiments relate to a method, system, and computer readable medium for detecting intracranial hemorrhage. According to one aspect, a method for detecting intracranial hemorrhage is provided. The method may include receiving, by a computer, data corresponding to a tomograph scan associated with a patient and extracting one or more slices from the received tomograph scan data. One or more adjacent slices may be determined for each of the extracted slices, and the extracted slices and the one or more adjacent slices may be grouped into one or more slabs. The computer may identify one or more features associated with the one or more slab and determine that a slab corresponding to the one or more identified features contains a feature associated with ICH.

According to another aspect, a computer system for detecting intracranial hemorrhage is provided. The computer system may include one or more processors, one or more computer-readable memories, one or more computer-readable tangible storage devices, and program instructions stored on at least one of the one or more storage devices for execution by at least one of the one or more processors via at least one of the one or more memories, whereby the computer system is capable of performing a method. The method may include receiving, by a computer, data corresponding to a tomograph scan associated with a patient and extracting one or more slices from the received tomograph scan data. One or more adjacent slices may be determined for each of the extracted slices, and the extracted slices and the one or more adjacent slices may be grouped into one or more slabs. The computer may identify one or more features associated with the one or more slab and determine that a slab corresponding to the one or more identified features contains a feature associated with ICH.

According to yet another aspect, a computer readable medium for detecting intracranial hemorrhage is provided. The computer readable medium may include one or more computer-readable storage devices and program instructions stored on at least one of the one or more tangible storage devices, the program instructions executable by a processor. The program instructions are executable by a processor for performing a method that may accordingly include receiving, by a computer, data corresponding to a tomograph scan associated with a patient and extracting one or more slices from the received tomograph scan data. One or more adjacent slices may be determined for each of the extracted slices, and the extracted slices and the one or more adjacent slices may be grouped into one or more slabs. The computer may identify one or more features associated with the one or more slab and determine that a slab corresponding to the one or more identified features contains a feature associated with ICH.

### BRIEF DESCRIPTION OF THE DRAWINGS

These and other objects, features and advantages will become apparent from the following detailed description of illustrative embodiments, which is to be read in connection with the accompanying drawings. The various features of the drawings are not to scale as the illustrations are for clarity in facilitating one skilled in the art in understanding this disclosure in conjunction with the detailed description. In the drawings:

FIG. 1 illustrates a networked computer environment according to at least one embodiment;

FIG. 2 is a block diagram of a program that detects intracranial hemorrhage, according to at least one embodiment;

FIG. 3 is a functional block diagram of a feature transform filter as depicted in FIG. 2, according to at least one embodiment;

FIG. 4 is an operational flowchart illustrating the steps carried out by a program that detects intracranial hemorrhage, according to at least one embodiment;

FIG. 5 is a block diagram of internal and external components of computers and servers depicted in FIG. 1 according to at least one embodiment;

FIG. 6 is a block diagram of an illustrative cloud computing environment including the computer system depicted in FIG. 1, according to at least one embodiment; and

FIG. 7 is a block diagram of functional layers of the illustrative cloud computing environment of FIG. 6, according to at least one embodiment.

### DETAILED DESCRIPTION

Detailed embodiments of the claimed structures and methods are disclosed herein; however, it can be understood that the disclosed embodiments are merely illustrative of the claimed structures and methods that may be embodied in various forms. Aspects of this disclosure may, however, be embodied in many different forms and should not be construed as limited to the exemplary embodiments set forth herein. Rather, these exemplary embodiments are provided so that this disclosure will be thorough and complete and will fully convey the scope of this disclosure to those skilled in the art. In the description, details of well-known features and techniques may be omitted to avoid unnecessarily obscuring the presented embodiments.

Embodiments relate generally to the field of medicine, and more particularly to detecting intracranial hemorrhage. The following described exemplary embodiments provide a system, method and program product to, among other things, predict whether adjacent two-dimensional (2D) computed tomography (CT) slices contain a pattern associated with intracranial hemorrhage (ICH). Some embodiments have the capacity to improve the field of medicine by allowing for the use of deep neural networks to augment traditional medical clinical data in diagnosing ICH. Thus, the computer-implemented method, computer system, and computer readable medium disclosed herein may, among other things, be used to determine a correlation between adjacent 2D CT slices, circumvent the intensive computations required for three-dimensional (3D) deep convolutional neural networks (DCNNs), and mitigate the impacts of data imbalance and labelling errors.

As previously described, ICH is a critical condition resulting from bleeding within the skull. ICH accounts for about two million strokes worldwide, and prompt diagnosis is required in order to optimize patient outcomes. Non-contrast CT scans of a patient's head are used for initial imaging in cases of head trauma or stroke-like symptoms. However, CT scans are inherently 3D images. Thus, neural networks may require large amounts of computing power to process and analyze 3D CT scan images. Streamlining the workflow of interpreting a head CT scan by automating the initial triage process may have the potential to substantially decrease the time for diagnosis and may expedite treatment. This may, in turn, decrease morbidity and mortality as a result of stroke and head injury. Automated head CT scan triage systems may be used to automatically manage the priority for interpretation of imaging studies with presumed ICH and help optimize radiology workflow.

2D DCNNs may be used to detect ICH in CT images. However, since CT images are inherently 3D, 2D DCNNs may be unable to factor in the correlations between 2D CT slices. Therefore, the performance of head CT triage systems based on 2D DCNN may not yield satisfactory results in clinical practice. To circumvent the limitations of 2D DCNN on detecting ICH in 3D CT images, 3D DCNNs may be used for head CT triage systems. However, although 3D DCNNs may be suitable for analyzing 3D CT images, such 3D DCNNs are computationally intensive to run. For example, due to limited graphics processing unit (GPU) memory, a batch size may, for example, only be set to a size of one for training 3D DCNNs. Additionally, 3D DCNNs may use a much smaller number of training data points than 2D DCNNs. Thus, 3D DCNNs may be limited in their applications in clinical settings.

To avoid the restrictions of 2D and 3D DCNNs, it may be advantageous, therefore, to utilize a semi-3D DCNN that may take in a number of 2D head CT slices as inputs and may output the ICH detection on CT images, such that the computation may be comparable to processing 2D images. The correlations between adjacent CT slices may be taken into consideration. Thus, automatic triage of head imaging studies using computer algorithms may have the potential to detect ICH earlier, ultimately leading to improved clinical outcomes. By using a deep learning system to automatically detect acute ICH based on non-contrast head computed tomography (CT) images, a semi-3D deep convolutional neural network (DCNN) may be used to analyze CT images, so that the limitations of 3D DCNN, such as computational intensity, data availability, and the “curse of dimensionality” can be avoided. Moreover, the loss function of the semi-3D DCNN may be modified to address data imbalance and labeling errors in order to circumvent the computational limitation of 3D DCNNs and achieve radiologist-level performance in ICH detection.

Aspects are described herein with reference to flowchart illustrations and/or block diagrams of methods, apparatus (systems), and computer readable media according to certain embodiments. It will be understood that each block of the flowchart illustrations and/or block diagrams, and combinations of blocks in the flowchart illustrations and/or block diagrams, can be implemented by computer readable program instructions.

The following described exemplary embodiments provide a system, method and program product that detects and diagnose intracranial hemorrhage in patients. According to the present embodiment, this detection may be provided through analysis of CT image data through deep learning to detect patterns associated with intracranial hemorrhage. Based on the detection of these patterns, the intracranial hemorrhage may be diagnosed and treated.

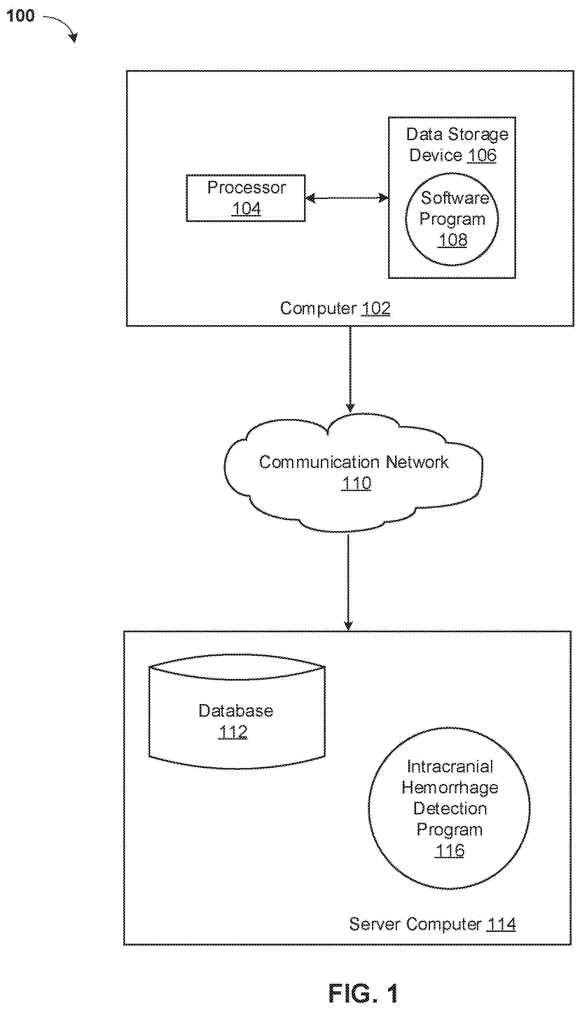

Referring now to FIG. 1, a functional block diagram of a networked computer environment illustrating an intracranial hemorrhage detection system 100 (hereinafter “system”) for improved detection of intracranial hemorrhage is shown. It should be appreciated that FIG. 1 provides only an illustration of one implementation and does not imply any limitations with regard to the environments in which different embodiments may be implemented. Many modifications to the depicted environments may be made based on design and implementation requirements.

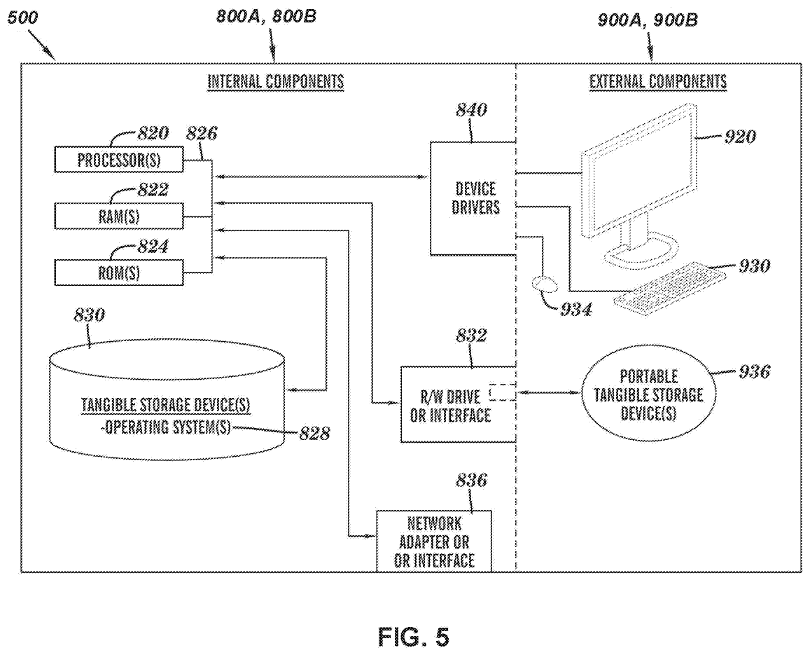

The system 100 may include a computer 102 and a server computer 114. The computer 102 may communicate with the server computer 114 via a communication network 110 (hereinafter “network”). The computer 102 may include a processor 104 and a software program 108 that is stored on a data storage device 106 and is enabled to interface with a user and communicate with the server computer 114. As will be discussed below with reference to FIG. 5 the computer 102 may include internal components 800A and external components 900A, respectively, and the server computer 114 may include internal components 800B and external components 900B, respectively. The computer 102 may be, for example, a mobile device, a telephone, a personal digital assistant, a netbook, a laptop computer, a tablet computer, a desktop computer, or any type of computing devices capable of running a program, accessing a network, and accessing a database.

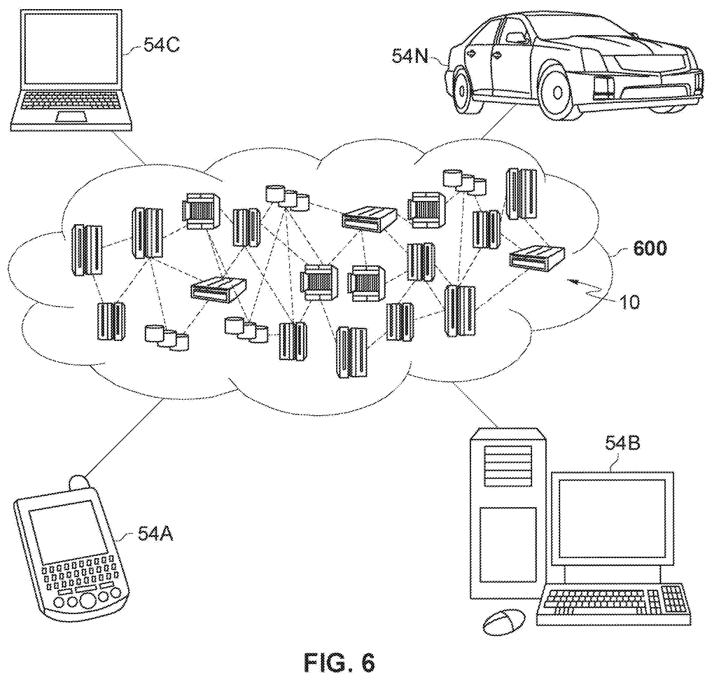

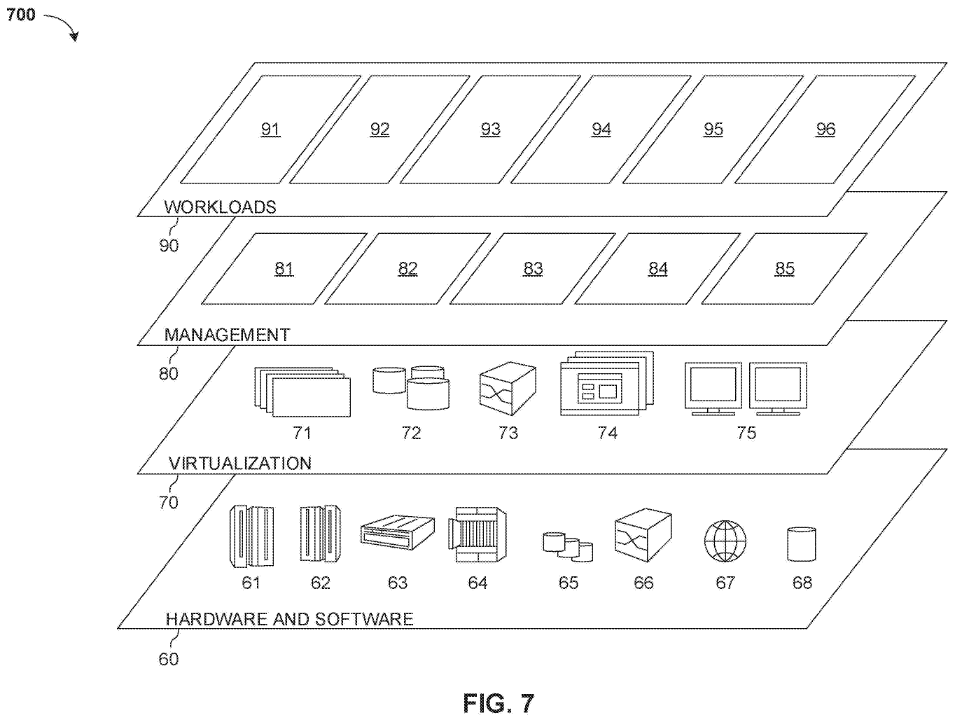

The server computer 114 may also operate in a cloud computing service model, such as Software as a Service (SaaS), Platform as a Service (PaaS), or Infrastructure as a Service (IaaS), as discussed below with respect to FIGS. 6 and 7. The server computer 114 may also be located in a cloud computing deployment model, such as a private cloud, community cloud, public cloud, or hybrid cloud.

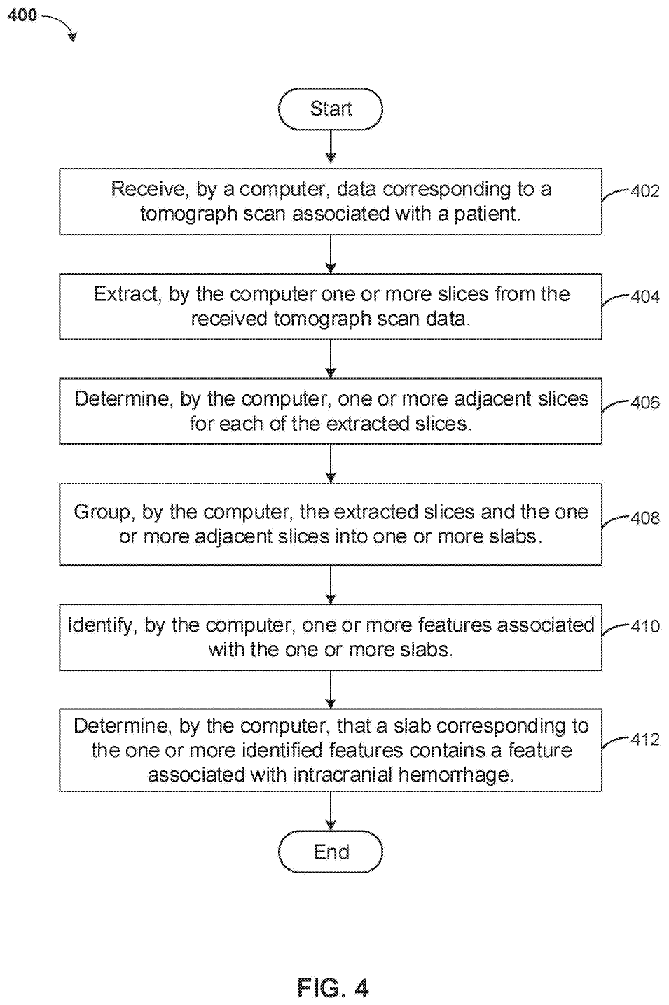

The server computer 114, which may be used for detecting, diagnosing, and notifying a user of intracranial hemorrhage is enabled to run an Intracranial Hemorrhage Detection Program 116 (hereinafter “program”) that may interact with a database 112. The Intracranial Hemorrhage Detection Program method is explained in more detail below with respect to FIG. 4. In one embodiment, the computer 102 may operate as an input device including a user interface while the program 116 may run primarily on server computer 114. In an alternative embodiment, the program 116 may run primarily on one or more computers 102 while the server computer 114 may be used for processing and storage of data used by the program 116. It should be noted that the program 116 may be a standalone program or may be integrated into a larger intracranial hemorrhage detection program.

It should be noted, however, that processing for the program 116 may, in some instances be shared amongst the computers 102 and the server computers 114 in any ratio. In another embodiment, the program 116 may operate on more than one computer, server computer, or some combination of computers and server computers, for example, a plurality of computers 102 communicating across the network 110 with a single server computer 114. In another embodiment, for example, the program 116 may operate on a plurality of server computers 114 communicating across the network 110 with a plurality of client computers. Alternatively, the program may operate on a network server communicating across the network with a server and a plurality of client computers.

The network 110 may include wired connections, wireless connections, fiber optic connections, or some combination thereof. In general, the network 110 can be any combination of connections and protocols that will support communications between the computer 102 and the server computer 114. The network 110 may include various types of networks, such as, for example, a local area network (LAN), a wide area network (WAN) such as the Internet, a telecommunication network such as the Public Switched Telephone Network (PSTN), a wireless network, a public switched network, a satellite network, a cellular network (e.g., a fifth generation (5G) network, a long-term evolution (LTE) network, a third generation (3G) network, a code division multiple access (CDMA) network, etc.), a public land mobile network (PLMN), a metropolitan area network (MAN), a private network, an ad hoc network, an intranet, a fiber optic-based network, or the like, and/or a combination of these or other types of networks.

The number and arrangement of devices and networks shown in FIG. 1 are provided as an example. In practice, there may be additional devices and/or networks, fewer devices and/or networks, different devices and/or networks, or differently arranged devices and/or networks than those shown in FIG. 1. Furthermore, two or more devices shown in FIG. 1 may be implemented within a single device, or a single device shown in FIG. 1 may be implemented as multiple, distributed devices. Additionally, or alternatively, a set of devices (e.g., one or more devices) of system 100 may perform one or more functions described as being performed by another set of devices of system 100.

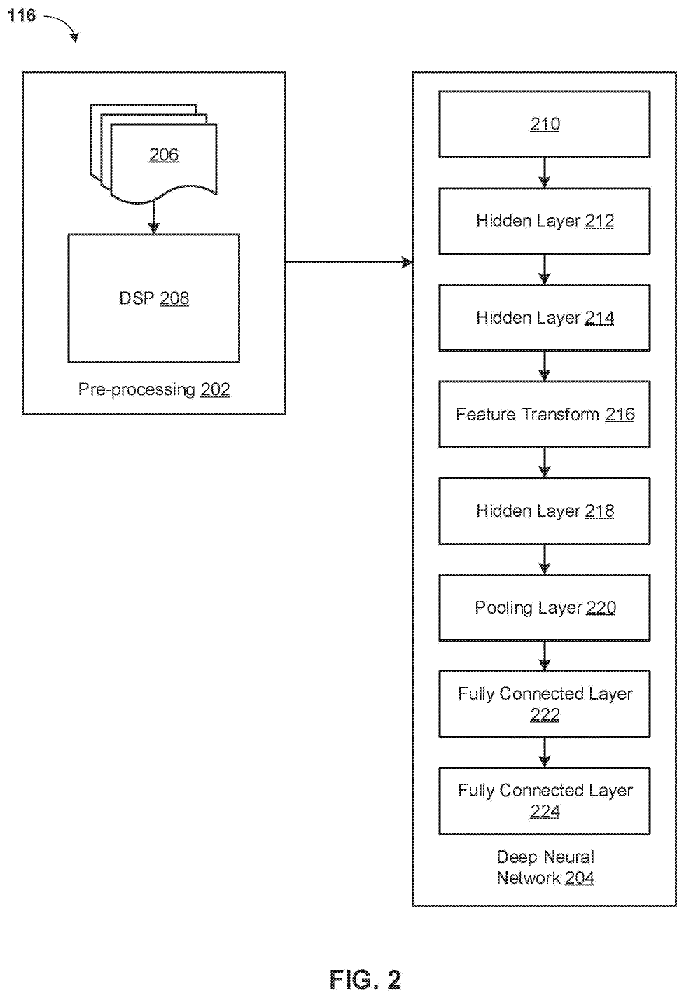

Referring to FIG. 2, a block diagram of an Intracranial Hemorrhage Detection Program 116 is depicted. FIG. 2 may be described with the aid of the exemplary embodiments depicted in FIG. 1. According to one or more embodiments, the Intracranial Hemorrhage Detection Program 116 may be located on the computer 102 (FIG. 1) or on the server computer 114 (FIG. 1). The Intracranial Hemorrhage Detection Program 116 may accordingly include, among other things, a pre-processing module 202 and a deep neural network 204. The pre-processing module 202 may contain a digital signal processing (DSP) module 208 and may be configured to retrieve data 206. According to one embodiment, the data 206 may be retrieved from the data storage device 106 (FIG. 1) on the computer 102. In an alternative embodiment, the data 206 may be retrieved from the database 112 (FIG. 1) on the server computer 114. The data 206 may include, among other things, CT images collected from a patient. The CT images may have, among other things, different sizes and windows settings. Thus, the preprocessing module 202 may crop the CT images so that only portions of the images corresponding to a patient's head are analyzed. The preprocessing module 202 may resize the cropped CT images. For example, the CT images may be resized to a size of 256 by 256 pixels. The pixel values of the images may be converted into Hounsfield units and may be filtered to specific values, such as, for example, between 0 and 130. Calcifications present within the images may also be considered when the window settings are chosen.

The DSP module 208 may extract one or more 2D CT image slices from the CT data. After preprocessing, adjacent CT slices may be grouped by the DSP module into one or more slabs, in which each slice is used as a channel of the input to a DCNN. It may be appreciated that slabs may be comprised of any number of adjacent CT slices. For example, in the case of a four-slice slab, the slab may considered positive for ICH if patterns associated with ICH are present within the 2^{nd} and/or 3^{rd} slices. Due to the data imbalance of head CT images, such as the fact that images with ICH features may occur less frequently than normal CT images and that certain types of ICH (e.g. epidural hemorrhages) may occur less frequently than other types of ICH (e.g. intracerebral hemorrhages), oversampling, sample weights and a focal loss function may be used to mitigate the data imbalance effects. Since the head CT slices may be manually labelled, label smoothing and smooth truncated loss function may be employed by the DSP module 208 to address possible labelling errors. Since the CT images may be imbalanced, the focal loss function may be used by the DSP module 208 to alleviate the impact of data imbalance, such that the minority data points may be assigned with larger weights in the loss function. Additionally, label smoothing and smooth truncated loss function may be utilized by the DSP module 208 to mitigate the effects of labelling errors. The DSP module 208 may also apply data cleaning and filtering to the data 206 for better processing by the deep neural network 204.

The deep neural network 204 may include, among other things, an input matrix 210; one or more hidden layers 212, 214, and 218; a feature transform layer 216; a pooling layer 220; and one or more connected layers 222 and 224. It may be appreciated that FIG. 2 depicts only one implementation of a deep neural network 204, and that the deep neural network 204 is not limited to these exact layers and order of layers. The deep neural network 204 may contain any number of layers in any order, including adding or omitting any of the depicted layers.

The input matrix 210 may, for example, be a two-dimensional matrix with dimensions n by k, whereby n may be a number of CT slices for analysis and k−1 may be a number of adjacent CT slices for each of the CT slices. For example, if 64 CT slices were to be analyzed with three adjacent CT slices for each of the 64 CT slices, the input matrix 210 would have a size of 64 by 4. However, it may be appreciated that n and k may be any values that may be selected based on available computation power, such that more neighborhood information may be kept for each CT slice for larger k values.

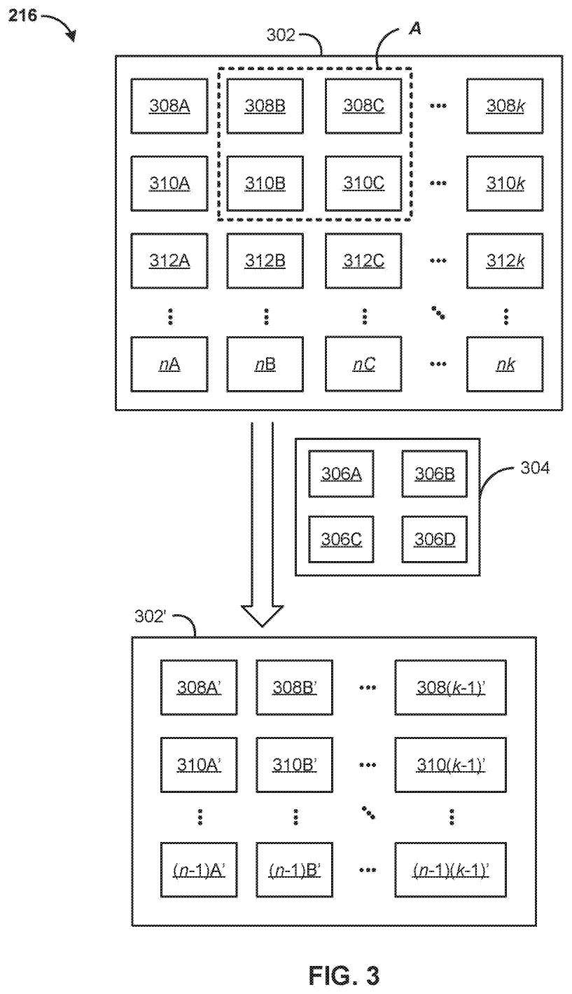

The feature transform layer 216 may be used to extract one or more features. The feature transform layer 216 is described in further detail with respect to FIG. 3. While only one feature transform layer 216 is depicted, it may be appreciated that the deep neural network 204 may contain additional feature transform layers 216 that may be applied to the data 206 in series or in parallel. The one or more hidden layers 212, 214, and 218 may be used to further process the data into a form usable by the deep neural network 204. The pooling layer 220 may be used to aggregate one or more features and down-sample the data analyzed for ease of identifying one or more features. The pooling layer 220 may apply a max-pooling strategy, an average-pooling strategy, or other pooling methods. The first fully connected layer 222 may be used, for example, to classify the aggregated features and to compare the features to one or more patterns. The patterns may be developed through deep learning, such that no human intervention may be present in the creation of the patterns. The second fully connected layer 224 may be used to classify whether the data 206 contains a pattern associated with intracranial hemorrhage by analyzing the output of the first fully connected layer 222. The second fully connected layer 224 may, for example, apply an indicator function to the data, such as outputting a “1” if the data contains a pattern associated with intracranial hemorrhage and outputting a “0” if the data does not. The deep neural network 204 may make an identification that ICH patterns are present within the CT data and may transmit this determination to a user, so that the user may, among other things, make any relevant diagnoses.

Referring now to FIG. 3, a function block diagram of an exemplary feature transform layer 216 is depicted, according to one or more embodiments. Feature transform layer 216 may contain a matrix 302 and a convolutional filter 304. By way of example and not of limitation, the convolutional filter 304 is depicted as a 2-by-2 matrix having four elements 306A-D. However, it may be appreciated that the convolutional filter 304 can be substantially any size with any number of elements. The matrix 302 may be, for example, a two-dimensional matrix having dimensions n by k, whereby n represents a number of CT slices for analysis and k−1 represents a number of adjacent slices. Thus, slices 308A, 310A, and 312A through nA may be stored within the first column of matrix 302. Additionally, adjacent slices 308B-k, 310B-k, 312B-k, and nB-k associated with each of slices 308A, 310A, 312A, and nA, respectively, may be stored in columns two through k of matrix 302. For example, where slices 308A, 310A, and 312A correspond to adjacent slices, it may be appreciated that slices 308A, 310B, and 312C may be the same, substantially the same, or similar. The convolutional filter 304 may be applied to any or all of the component submatrices (e.g., submatrix A containing slices 308B, 308C, 310B, and 310C) of the matrix 302 having the same, substantially the same, or similar size as the convolutional filter 304. The matrix 302′ may be generated as a result of calculating the scalar (i.e., dot) product of each of the component submatrices of the matrix 302 and the convolutional filter 304. For example, 308B′ may be the dot product of submatrix A and the convolutional filter 304.

Referring now to FIG. 4, an operational flowchart 400 illustrating the steps carried out by a program that detects intracranial hemorrhage is depicted. FIG. 4 may be described with the aid of FIGS. 1, 2, and 3. As previously described, the Intracranial Hemorrhage Detection Program 116 (FIG. 1) may quickly and effectively detect intracranial hemorrhage.

At 402, data corresponding to a tomograph scan associated with a patient is received by a computer. The tomograph scan data may include, among other thing, a computed tomography (CT) scan, a magnetic resonance imaging (MRI) scan, a functional magnetic resonance imaging (fMRI) scan, or a positron emission tomography (PET) scan. The tomograph scan data may include images corresponding to a patient's head. In operation, the Intracranial Hemorrhage Detection Program 116 (FIG. 1) may reside on the computer 102 (FIG. 1) or on the server computer 114 (FIG. 1). The Intracranial Hemorrhage Detection Program 116 may receive data 206 (FIG. 2) over the communication network 110 (FIG. 1) or may retrieve the data 206 from the database 112 (FIG. 1)

At 404, one or more slices from the received tomograph scan data are extracted by the computer. The tomograph scan data may, for example, be received in the form of a 3D tomograph image comprised of one or more 2D image slices. Thus, extracting one or more 2D image slices from the 3D image may allow for a qualitative analysis of the CT data by allowing a comparison between adjacent slices. A number, n, of CT slices may be stored in a column of an n by k two-dimensional matrix. In operation, the DSP module 208 (FIG. 2) may identify one or more 2D CT image slices from among the data 206 (FIG. 2). The DSP module 208 may, for example, store the data 206 in the first column of the input matrix 210 (FIG. 2).

At 406, one or more adjacent slices for each of the extracted slices are determined by the computer. The adjacent CT slices may, among other things provide data for each of the CT slices to be analyzed and may, for example, allow for the detection of unintuitive patterns to assist in diagnosing and treating ICH. The adjacent CT slices may be stored within the second and subsequent columns matrix. There may be, for example, k−1 adjacent CT slices for each of the n CT slices that may be stored in columns 2 through k of the two-dimensional matrix. In operation, the DSP module 208 (FIG. 2) may identify a number of adjacent CT slices for each of the CT slices present within the data 206 (FIG. 2). The DSP module 208 may store this information in the second and subsequent columns of input matrix 210 (FIG. 2)

At 408, the extracted slices and the one or more adjacent slices are grouped into one or more slabs by the computer. Because one or more convolutional filters may be applied to the data, it may be advantageous, for example, to down-sample the data by aggregating features in order to make processing the data more manageable and save on computing resources. In operation, the feature transform layer 216 (FIG. 2) may apply a convolutional filter 304 (FIG. 3) to the matrix 302 (FIG. 3). The convolutional filter 304 may be, for example, a size 2-by-2 array and may be applied to matrix 302 by calculating a dot product for each of the component 2-by-2 arrays of the matrix 302. Thus, a matrix 302′ (FIG. 3) having a size (k−1)-by-(n−1) may be produced as a result of applying the convolutional filter 304 to the matrix 302. It may be appreciated that one or more convolutional filters 304 may be applied to the matrix 302 simultaneously, yielding one or more matrices 302′. These matrices 302′ may be appended to one another by, for example, the hidden layer 218 (FIG. 2) to create a higher-order multi-dimensional array. The pooling layer 220 (FIG. 2) may apply one or more pooling strategies to the matrix 302′, such as max-pooling or average-pooling. For example, the pooling layer 220 may apply max-pooling to the matrix 302′ such that the maximum value present in each non-overlapping 2-by-2 component submatrix of the matrix 302′ may be placed into a cell in a matrix having an approximate size (n−1)/2-by-(k−1)/2.

At 410, one or more features associated with the one or more slabs are identified by the computer. After the features have been aggregated, the system may identify one or more patterns from among the features, such as patterns associated with ICH. In operation, the first fully connected layer 222 (FIG. 2) of the deep neural network 204 (FIG. 2) may analyze the down-sampled matrix output by the pooling layer 220 (FIG. 2) to determine if any patterns consistent with ICH are present within the data 206 (FIG. 2). If any patterns are detected, the system may accordingly classify them based on the presence of such patterns.

At 412, the computer determines that a slab corresponding to the one or more identified features contains a feature associated with ICH. After determining the presence of one or more patterns present within the data, the computer may, among other things, determine whether one or more of these patterns correspond to ICH. By learning, through patterns in the data, whether the data contains ICH, identification of such a condition can be made without human intervention and without bias in the development of the model. In operation, the second fully connected layer 224 (FIG. 2) of the deep neural network 204 (FIG. 2) may apply a filter to the output of the first fully connected layer 222 (FIG. 2) to determine whether there exists a pattern in the data 206 that corresponds to ICH. The second fully connected layer 224 may output, for example, a “1” if it determines that an ICH pattern may be present with the data 206. The second fully connected layer 224 may additionally output, for example, a “0” if it determines that an ICH pattern may not be present within the data 206.

It may be appreciated that FIG. 4 provides only an illustration of one implementation and does not imply any limitations with regard to how different embodiments may be implemented. Many modifications to the depicted environments may be made based on design and implementation requirements.

FIG. 5 is a block diagram 500 of internal and external components of computers depicted in FIG. 1 in accordance with an illustrative embodiment. It should be appreciated that FIG. 5 provides only an illustration of one implementation and does not imply any limitations with regard to the environments in which different embodiments may be implemented. Many modifications to the depicted environments may be made based on design and implementation requirements.

Computer 102 (FIG. 1) and server computer 114 (FIG. 1) may include respective sets of internal components 800A,B and external components 900A,B illustrated in FIG. 5. Each of the sets of internal components 800 include one or more processors 820, one or more computer-readable RAMs 822 and one or more computer-readable ROMs 824 on one or more buses 826, one or more operating systems 828, and one or more computer-readable tangible storage devices 830.

Processor 820 is implemented in hardware, firmware, or a combination of hardware and software. Processor 820 is a central processing unit (CPU), a graphics processing unit (GPU), an accelerated processing unit (APU), a microprocessor, a microcontroller, a digital signal processor (DSP), a field-programmable gate array (FPGA), an application-specific integrated circuit (ASIC), or another type of processing component. In some implementations, processor 820 includes one or more processors capable of being programmed to perform a function. Bus 826 includes a component that permits communication among the internal components 800A,B.

The one or more operating systems 828, the software program 108 (FIG. 1) and the Intracranial Hemorrhage Detection Program 116 (FIG. 1) on server computer 114 (FIG. 1) are stored on one or more of the respective computer-readable tangible storage devices 830 for execution by one or more of the respective processors 820 via one or more of the respective RAMs 822 (which typically include cache memory). In the embodiment illustrated in FIG. 5, each of the computer-readable tangible storage devices 830 is a magnetic disk storage device of an internal hard drive. Alternatively, each of the computer-readable tangible storage devices 830 is a semiconductor storage device such as ROM 824, EPROM, flash memory, an optical disk, a magneto-optic disk, a solid state disk, a compact disc (CD), a digital versatile disc (DVD), a floppy disk, a cartridge, a magnetic tape, and/or another type of non-transitory computer-readable tangible storage device that can store a computer program and digital information.

Each set of internal components 800A,B also includes a R/W drive or interface 832 to read from and write to one or more portable computer-readable tangible storage devices 936 such as a CD-ROM, DVD, memory stick, magnetic tape, magnetic disk, optical disk or semiconductor storage device. A software program, such as the software program 108 (FIG. 1) and the Intracranial Hemorrhage Detection Program 116 (FIG. 1) can be stored on one or more of the respective portable computer-readable tangible storage devices 936, read via the respective R/W drive or interface 832 and loaded into the respective hard drive 830.

Each set of internal components 800A,B also includes network adapters or interfaces 836 such as a TCP/IP adapter cards; wireless Wi-Fi interface cards; or 3G, 4G, or 5G wireless interface cards or other wired or wireless communication links. The software program 108 (FIG. 1) and the Intracranial Hemorrhage Detection Program 116 (FIG. 1) on the server computer 114 (FIG. 1) can be downloaded to the computer 102 (FIG. 1) and server computer 114 from an external computer via a network (for example, the Internet, a local area network or other, wide area network) and respective network adapters or interfaces 836. From the network adapters or interfaces 836, the software program 108 and the Intracranial Hemorrhage Detection Program 116 on the server computer 114 are loaded into the respective hard drive 830. The network may comprise copper wires, optical fibers, wireless transmission, routers, firewalls, switches, gateway computers and/or edge servers.

Each of the sets of external components 900A,B can include a computer display monitor 920, a keyboard 930, and a computer mouse 934. External components 900A,B can also include touch screens, virtual keyboards, touch pads, pointing devices, and other human interface devices. Each of the sets of internal components 800A,B also includes device drivers 840 to interface to computer display monitor 920, keyboard 930 and computer mouse 934. The device drivers 840, R/W drive or interface 832 and network adapter or interface 836 comprise hardware and software (stored in storage device 830 and/or ROM 824).

It is understood in advance that although this disclosure includes a detailed description on cloud computing, implementation of the teachings recited herein are not limited to a cloud computing environment. Rather, some embodiments are capable of being implemented in conjunction with any other type of computing environment now known or later developed.

Cloud computing is a model of service delivery for enabling convenient, on-demand network access to a shared pool of configurable computing resources (e.g. networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure comprising a network of interconnected nodes.

Referring to FIG. 6, illustrative cloud computing environment 600 is depicted. As shown, cloud computing environment 600 comprises one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Cloud computing nodes 10 may communicate with one another. They may be grouped (not shown) physically or virtually, in one or more networks, such as Private, Community, Public, or Hybrid clouds as described hereinabove, or a combination thereof. This allows cloud computing environment 600 to offer infrastructure, platforms and/or software as services for which a cloud consumer does not need to maintain resources on a local computing device. It is understood that the types of computing devices 54A-N shown in FIG. 6 are intended to be illustrative only and that cloud computing nodes 10 and cloud computing environment 600 can communicate with any type of computerized device over any type of network and/or network addressable connection (e.g., using a web browser).

Referring to FIG. 7, a set of functional abstraction layers 700 provided by cloud computing environment 600 (FIG. 6) is shown. It should be understood in advance that the components, layers, and functions shown in FIG. 7 are intended to be illustrative only and embodiments of the disclosure are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may comprise application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and Intracranial Hemorrhage Detection 96. Intracranial Hemorrhage Detection 96 may detect and classify patterns associated with intracranial hemorrhage in a patient.

Some embodiments may relate to a system, a method, and/or a computer readable medium at any possible technical detail level of integration. The computer readable medium may include a computer-readable non-transitory storage medium (or media) having computer readable program instructions thereon for causing a processor to carry out operations.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program code/instructions for carrying out operations may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects or operations.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer readable media according to various embodiments. In this regard, each block in the flowchart or block diagrams may represent a module, segment, or portion of instructions, which comprises one or more executable instructions for implementing the specified logical function(s). The method, computer system, and computer readable medium may include additional blocks, fewer blocks, different blocks, or differently arranged blocks than those depicted in the Figures. In some alternative implementations, the functions noted in the blocks may occur out of the order noted in the Figures. For example, two blocks shown in succession may, in fact, be executed concurrently or substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

It will be apparent that systems and/or methods, described herein, may be implemented in different forms of hardware, firmware, or a combination of hardware and software. The actual specialized control hardware or software code used to implement these systems and/or methods is not limiting of the implementations. Thus, the operation and behavior of the systems and/or methods were described herein without reference to specific software code—it being understood that software and hardware may be designed to implement the systems and/or methods based on the description herein.

No element, act, or instruction used herein should be construed as critical or essential unless explicitly described as such. Also, as used herein, the articles “a” and “an” are intended to include one or more items, and may be used interchangeably with “one or more.” Furthermore, as used herein, the term “set” is intended to include one or more items (e.g., related items, unrelated items, a combination of related and unrelated items, etc.), and may be used interchangeably with “one or more.” Where only one item is intended, the term “one” or similar language is used. Also, as used herein, the terms “has,” “have,” “having,” or the like are intended to be open-ended terms. Further, the phrase “based on” is intended to mean “based, at least in part, on” unless explicitly stated otherwise.

The descriptions of the various aspects and embodiments have been presented for purposes of illustration, but are not intended to be exhaustive or limited to the embodiments disclosed. Even though combinations of features are recited in the claims and/or disclosed in the specification, these combinations are not intended to limit the disclosure of possible implementations. In fact, many of these features may be combined in ways not specifically recited in the claims and/or disclosed in the specification. Although each dependent claim listed below may directly depend on only one claim, the disclosure of possible implementations includes each dependent claim in combination with every other claim in the claim set. Many modifications and variations will be apparent to those of ordinary skill in the art without departing from the scope of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method of detecting intracranial hemorrhage (ICH), comprising:
receiving, by a computer, data corresponding to a tomograph scan associated with a patient;
extracting, by the computer one or more slices from the received tomograph scan data;
determining, by the computer, one or more adjacent slices for each of the extracted slices;
grouping, by the computer, the extracted slices and the one or more adjacent slices into one or more slabs;
identifying one or more features associated with the one or more slabs; and
determining, by the computer, that a slab corresponding to the one or more identified features contains a feature associated with ICH.

2. The method of claim 1, wherein the slices are stored in a two dimensional array corresponding to the one or more extracted slices and the one or more determined adjacent slices for each of the extracted slices.

3. The method of claim 2, wherein the features are identified in response to generating a multi-dimensional array in response to applying one or more convolutional filter layers to the two-dimensional array.

4. The method of claim 3, wherein determining that the slab corresponding to the one or more identified features contains the feature associated with ICH comprises applying a fully connected layer to the multi-dimensional array.

5. The method of claim 1, further comprising:
transmitting, by the computer, to a user, the determination of the slab corresponding to the one or more identified features containing the feature associated with ICH.

6. The method of claim 1, wherein one or more pixels associated with the extracted slices are converted to Hounsfield units corresponding to features associated with the pixels.

7. The method of claim 1, wherein the identifying the one or more features comprises applying a max-pooling layer to the one or more slices and the one or more adjacent slices.

8. The method of claim 1, wherein the identifying the one or more features comprises applying an average-pooling layer to the one or more slices and the one or more adjacent slices.

9. The method of claim 1, further comprising:
assigning, by the computer, a weight value to the slices based on a focal loss function.

10. The method of claim 1, wherein the tomograph scan comprises one or more of: a computed tomography (CT) scan, a magnetic resonance imaging (MRI) scan, a functional magnetic resonance imaging (fMRI) scan, or a positron emission tomography (PET) scan.

11. A computer system for detecting intracranial hemorrhage (ICH), the computer system comprising:
one or more computer-readable non-transitory storage media configured to store computer program code; and
one or more computer processors configured to access said computer program code and operate as instructed by said computer program code, said computer program code including:
receiving code configured to cause the one or more computer processors to receive data corresponding to a tomograph scan associated with a patient;
extracting code configured to cause the one or more computer processors to extract one or more slices from the received tomograph scan data;
determining code configured to cause the one or more computer processors to determine one or more adjacent slices for each of the extracted slices;
grouping code configured to cause the one or more computer processors to group the extracted slices and the one or more adjacent slices into one or more slabs;
identifying code configured to cause the one or more computer processors to identify one or more features associated with the one or more slabs; and
determining code configured to cause the one or more computer processors to determine that a slab corresponding to the one or more identified features contains a feature associated with ICH.

12. The computer system of claim 11, wherein the slices are stored in a two dimensional array corresponding to the one or more extracted slices and the one or more determined adjacent slices for each of the extracted slices.

13. The computer system of claim 12, wherein the features are identified in response to generating a multi-dimensional array in response to applying one or more convolutional filter layers to the two-dimensional array.

14. The computer system of claim 13, wherein determining code configured to cause the one or more computer processors to determine that a slab corresponding to the one or more identified features contains a feature associated with ICH comprises applying code configured to cause the one or more computer processors to apply a fully connected layer to the multi-dimensional array.

15. The computer system of claim 11, further comprising:
transmitting code configured to cause the one or more computer processors to transmit, to a user, the determination of the slab corresponding to the one or more identified features containing the feature associated with ICH.

16. The computer system of claim 11, wherein one or more pixels associated with the extracted slices are converted to Hounsfield units corresponding to features associated with the pixels.

17. The computer system of claim 11, wherein the identifying code comprises applying code to code configured to cause the one or more computer processors to apply a max-pooling layer to the one or more slices and the one or more adjacent slices.

18. The computer system of claim 11, wherein the identifying code comprises applying code configured to cause the one or more computer processors to apply an average-pooling layer to the one or more slices and the one or more adjacent slices.

19. The computer system of claim 11, further comprising:
assigning code configured to cause the one or more computer processors to assign a weight value to the slices based on a focal loss function.

20. A non-transitory computer readable medium having stored thereon a computer program for detecting intracranial hemorrhage (ICH), the computer program configured to cause one or more computer processors to:
receive data corresponding to a tomograph scan associated with a patient;
extract one or more slices from the received tomograph scan data;
determine one or more adjacent slices for each of the extracted slices;
group the extracted slices and the one or more adjacent slices into one or more slabs;
identify one or more features associated with the one or more slabs; and
determine that a slab corresponding to the one or more identified features contains a feature associated with ICH.
